(19) United States
(12) Patent Application Publication
(10) Pub. No.: US 2020/0246521 A1
(43) Pub. Date: Aug. 6, 2020

(54) EXTRACORPOREAL-CIRCULATION BLOOD PUMP DEVICE

(71) Applicant: TERUMO KABUSHIKI KAISHA, Tokyo (JP)

(72) Inventor: Toshihiko Nojiri, Kanagawa (JP)

(21) Appl. No.: 16/832,063

(22) Filed: Mar. 27, 2020

Related U.S. Application Data
(63) Continuation of application No. PCT/JP2018/037317, filed on Oct. 5, 2018.

(30) Foreign Application Priority Data
Oct. 6, 2017 (JP) 2017-195598

(51) Int. Cl. A61M 1/10

(52) U.S. Cl. CPC A61M 1/101; A61M 2209/088; A61M 2205/8206; A61M 1/1036

(57) ABSTRACT

An extracorporeal-circulation blood pump device (10) includes a pump body (22) to which a catheter (16) for removing blood from a patient (100) is connected, and which houses an impeller (14) in a rotatable manner; and a drive device (24) detachably mounted to the pump body (22) and rotating the impeller (14). The pump device (10) also has a wound covering sheet (30) to be attached to the body surface (102) of the patient (100). The wound covering sheet (30) can cover both the pump body (22) and at least a location where the catheter (16) is inserted into the body.

### CROSS REFERENCE TO RELATED APPLICATIONS

This application is a continuation of PCT Application No. PCT/JP2018/037317, filed Oct. 5, 2018, based on and claiming priority to Japanese Application No. 2017-195598, filed Oct. 6, 2017, both of which are incorporated herein by reference in their entirety.

### BACKGROUND OF THE INVENTION

The present invention relates to an extracorporeal-circulation blood pump device that allows blood of a patient to flow outside the body.

### BACKGROUND ART

An extracorporeal-circulation blood pump device is used as a blood circulation device for circulating blood in a pump oxygenator or an external ventricular assist device. For example, U.S. Pat. No. 5,316,440 discloses a pump device including a pump body enclosing an impeller in a rotatable manner and a drive device to which the pump body is mounted and which rotates the impeller. In this pump device, only the pump body which comes in contact with blood is disposable, so that an expensive motor can be reused.

Meanwhile, the extracorporeal-circulation blood pump device designed for a pump oxygenator as disclosed in U.S. Pat. No. 5,316,440 is large in size and heavy, so that it is often placed on a cart, etc. at a location some distance from a patient. As a result, a patient with reduced cardiac function who uses an extracorporeal-circulation blood pump device alone as an external ventricular assist device that assists the blood circulation of the patient outside the body has a significant restriction on daily movement and performance, which may affect, for example, postoperative rehabilitation. That is, in the technical field of extracorporeal-circulation blood pump devices, a device that is stably carried by a patient and enables the patient to act more freely is desirable.

Furthermore, in the extracorporeal-circulation blood pump device, a blood removal tube, a blood supply tube, etc. are inserted into the body of the patient from the outside of the body, and there is also a problem that bacteria, etc. easily enter the body (the body is easily infected with bacteria, etc.) through the locations where such tubes are inserted. In particular, if the tubes move relative to the body surface due to the movement of the patient, the location (wound) where the tubes are inserted increases, which may increase the possibility of infection.

### SUMMARY OF THE INVENTION

The present invention has been made to solve the above problems, and provides an extracorporeal-circulation blood pump device that can be stably and hygienically placed on the body surface of a patient with a simple configuration.

In order to achieve the above-mentioned object, the present invention provides an extracorporeal-circulation blood pump device comprising: a pump body to which a blood removal tube for removing blood from a patient and a blood supply tube for supplying the blood to the patient configured to be connected, and which houses an impeller having inside a body-side magnetic coupling section in a rotatable manner; and a drive device detachably mounted to the pump body and having inside a drive-side magnetic coupling section which includes a rotatable permanent magnet or a stator coil generating a rotating magnetic field, the impeller being rotatable by magnetic coupling between the body-side magnetic coupling section and the drive-side magnetic coupling section, the extracorporeal-circulation blood pump device further comprising a covering sheet configured to affix the pump body to a body surface of the patient, wherein the covering sheet extends radially outward with respect to the pump body and is capable of covering both the pump body and at least a location where the blood removal tube and the blood supply tube are inserted into the body, when being attached.

With the configuration described above, the extracorporeal-circulation blood pump device is stably and hygienically placed on the body surface of the patient by a simple configuration having the covering sheet. In particular, the covering sheet extends radially outward with respect to the pump body and can cover the location where the blood removal tube and the blood supply tube are inserted into the body and the pump body together, when being attached. Thus, the covering sheet can reliably prevent a portion where blood flows from being exposed. Therefore, the covering sheet can reliably prevent positional displacement of the extracorporeal-circulation blood pump device, the blood removal tube, and the blood supply tube, and can satisfactorily block the invasion of bacteria into the body.

It is preferable that the covering sheet is disposed between the pump body and the drive device when the pump body and the drive device are assembled together.

In the extracorporeal-circulation blood pump device, the covering sheet is disposed between the pump body and the drive device, so that the covering sheet is located near the pump body, the blood removal tube, and the blood supply tube, and can easily fix them tightly.

It is also preferable that the covering sheet has an opening in which the pump body or the drive device is disposed to pass therethrough, and one of the pump body and the drive device disposed in the opening so as to pass through the opening is mounted to the other in a detachable manner.

In the extracorporeal-circulation blood pump device, the pump body and the drive device can be attached to and detached from each other in the opening of the covering sheet, so that the pump body and the drive device can be firmly fixed to each other to prevent positional displacement.

It is still preferable that a hard cover made of a hard material is disposed in the opening so as to pass through the opening, and the covering sheet is integrated with the hard cover in an airtight manner.

The extracorporeal-circulation blood pump device enables assembling of the pump body and the drive device together with the hard cover interposed therebetween, thereby being capable of more reliably preventing positional displacement of the pump body, the drive device, and the covering sheet. Furthermore, if the covering sheet and the hard cover are airtightly integrated, a member such as the covering sheet can be fixed to the pump body or the drive device without any adhesive or the like, and thus, such a member can be easily replaced.

Here, the covering sheet may be fixed to the pump body.

In the extracorporeal-circulation blood pump device, the pump body and the covering sheet can be handled together, because the covering sheet is fixed to the pump body. Thus, the pump body can be quickly attached to the body surface.

It is preferable that the impeller is provided with a driven rotating structure section including the body-side magnetic coupling section, the impeller being supported so as to float and rotate in a radial direction by a dynamic pressure bearing formed between an outer periphery of the driven rotating structure section and an inner periphery of a housing that houses the driven rotating structure section, and the driven rotating structure section is held at a substantially fixed position in a direction of a rotation axis by a magnetic bearing formed by the body-side magnetic coupling section and the drive-side magnetic coupling section.

According to the above configuration, the impeller of the extracorporeal-circulation blood pump device can rotate, while floating, without contacting the housing, thereby being capable of circulating blood more safely by preventing blood destruction or coagulation due to friction or the like. In addition, a storage space for the bearing member is not required, whereby the pump body can be made more compact, and the manufacturing cost can be reduced.

It is also preferable that the pump body is placed in such a way that a rotation axis of the driven rotating structure section is parallel to the body surface, and the drive-side magnetic coupling section of the drive device is a permanent magnet that rotates around a rotation axis disposed coaxial with the rotation axis of the driven rotating structure section.

As a result, the extracorporeal-circulation blood pump device can be configured to rotate the impeller with a small diameter that is long in the direction of the rotation axis at a high speed, and enables the pump body to be placed on the body surface without increasing the height of the pump body protruding from the body surface.

Alternatively, it is preferable that the pump body is placed in such a way that a rotation axis of the driven rotating structure section is parallel to the body surface, and the drive-side magnetic coupling section of the drive device is a permanent magnet that rotates around a rotation axis perpendicular to the rotation axis of the driven rotating structure section.

Even in this case, the extracorporeal-circulation blood pump device can be configured to rotate the impeller with a small diameter that is long in the direction of the rotation axis at a high speed, and enables the pump body to be placed on the body surface without increasing the height of the pump body protruding from the body surface. Furthermore, the extracorporeal-circulation blood pump device enables the drive device to be mounted in a direction perpendicular to the body surface, which simplifies the mounting of the drive device to the pump body. Further, the pump body can be more easily and reliably covered with the covering sheet.

It is preferable that the extracorporeal-circulation blood pump device further comprises: a control unit that is electrically connected to the drive device and controls driving of the drive device; a battery capable of supplying electric power to the drive device and the control unit; and a storage bag or band capable of holding the control unit and the battery together in a portable manner.

The extracorporeal-circulation blood pump device enables the patient to move more easily by holding the control unit and the battery by the storage bag or band in a portable manner.

In order to achieve the above object, an extracorporeal-circulation blood pump device according to the present invention comprises: a pump body to which a blood removal tube for removing blood from a patient and a blood supply tube for supplying the blood to the patient are connected, and which houses an impeller having inside a body-side magnetic coupling section in a rotatable manner; a drive device detachably mounted to the pump body and having inside a drive-side magnetic coupling section which includes a rotatable permanent magnet or a stator coil generating a rotating magnetic field; and a covering sheet configured to affix the pump body to a body surface of the patient, wherein the covering sheet has an opening in which the pump body or the drive device is disposed to pass therethrough, and one of the pump body and the drive device disposed in the opening so as to pass through the opening is mounted to the other in a detachable manner.

With the configuration described above, the extracorporeal-circulation blood pump device is configured such that one of the pump body and the drive device disposed in the opening of the covering sheet so as to pass through the opening is mounted to the other in a detachable manner, whereby the pump body and the drive device can be assembled together with the covering sheet interposed therebetween. As a result, the covering sheet can easily affix one of the pump body and the drive device onto the body surface. That is, the extracorporeal-circulation blood pump device can be stably and hygienically placed on the body surface of the patient.

According to the present invention, the extracorporeal-circulation blood pump device is stably and hygienically placed on the body surface of a patient with a simple configuration.

### BRIEF DESCRIPTION OF THE DRAWINGS

FIG. 1 is an explanatory view schematically showing an extracorporeal-circulation blood pump device according to an embodiment of the present invention.

FIG. 2 is a plan view schematically showing a main part of the extracorporeal-circulation blood pump device in FIG. 1.

FIG. 3 is a side sectional view showing the main part of the extracorporeal-circulation blood pump device in FIG. 1.

FIG. 4A is a sectional view taken along a line IVA-IVA in FIG. 3, FIG. 4B is a sectional view taken along a line IVB-IVB in FIG. 3, and FIG. 4C is a sectional view taken along a line IVC-IVC in FIG. 3.

FIG. 5 is a side sectional view showing the operation of the extracorporeal-circulation blood pump device.

FIG. 6A is an exploded side sectional view showing an extracorporeal-circulation blood pump device according to a first modification, and FIG. 6B is a side sectional view showing an assembled state of the extracorporeal-circulation blood pump device shown in FIG. 6A.

FIG. 7A is an exploded side sectional view showing an extracorporeal-circulation blood pump device according to a second modification, and FIG. 7B is a side sectional view showing an assembled state of the extracorporeal-circulation blood pump device shown in FIG. 7A.

FIG. 8 is a side sectional view showing an extracorporeal-circulation blood pump device according to a third modification.

FIG. 9 is a side sectional view showing an extracorporeal-circulation blood pump device according to a fourth modification.

FIG. 10A is a sectional view taken along a line XA-XA in FIG. 9, and FIG. 10B is a sectional view taken along a line XB-XB in FIG. 9.

FIG. 11 is an exploded side sectional view showing an extracorporeal-circulation blood pump device according to a fifth modification.

FIG. 12A is a perspective view showing an extracorporeal-circulation blood pump device according to a sixth modification, and FIG. 12B is a perspective view of the pump body in FIG. 12A viewed from another angle.

FIG. 13A is a first sectional plan view showing a state where the pump body and the drive device in FIG. 12A are separated from each other, and FIG. 13B is a second sectional plan view showing a state where the pump body and the drive device in FIG. 12A are assembled together.

FIG. 14 is a perspective view showing a state where a wound covering sheet is put on the pump body in FIG. 12A.

FIG. 15A is a side sectional view showing an extracorporeal-circulation blood pump device according to a seventh modification, and FIG. 15B is a front sectional view of the extracorporeal-circulation blood pump device shown in FIG. 15A.

FIG. 16 is an exploded side sectional view showing the extracorporeal-circulation blood pump device shown in FIG. 15A.

FIG. 17 is a perspective view showing pole pieces in FIG. 15A.

FIG. 18 is a perspective view showing an extracorporeal-circulation blood pump device according to an eighth modification.

FIG. 19 is an exploded perspective view showing the extracorporeal-circulation blood pump device in FIG. 18.

FIG. 20 is side sectional view showing the extracorporeal-circulation blood pump device in FIG. 18.

FIG. 21 is a sectional view, taken along a line XXI-XXI in FIG. 20, showing magnetic coupling of the extracorporeal-circulation blood pump device.

FIG. 22 is an explanatory view schematically showing a first use example of an extracorporeal-circulation blood pump device.

FIG. 23 is an explanatory view schematically showing a second use example of an extracorporeal-circulation blood pump device.

### DETAILED DESCRIPTION OF PREFERRED EMBODIMENTS

Hereinafter, preferred embodiments of an extracorporeal-circulation blood pump device according to the present invention will be described in detail with reference to the accompanying drawings.

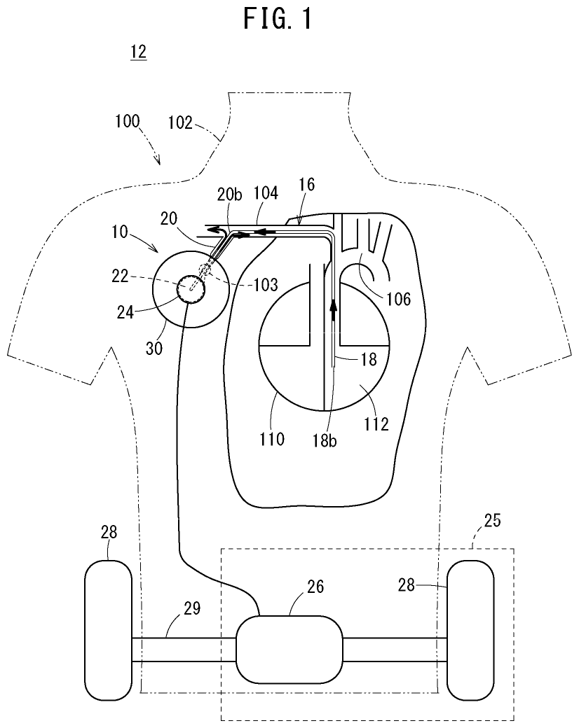

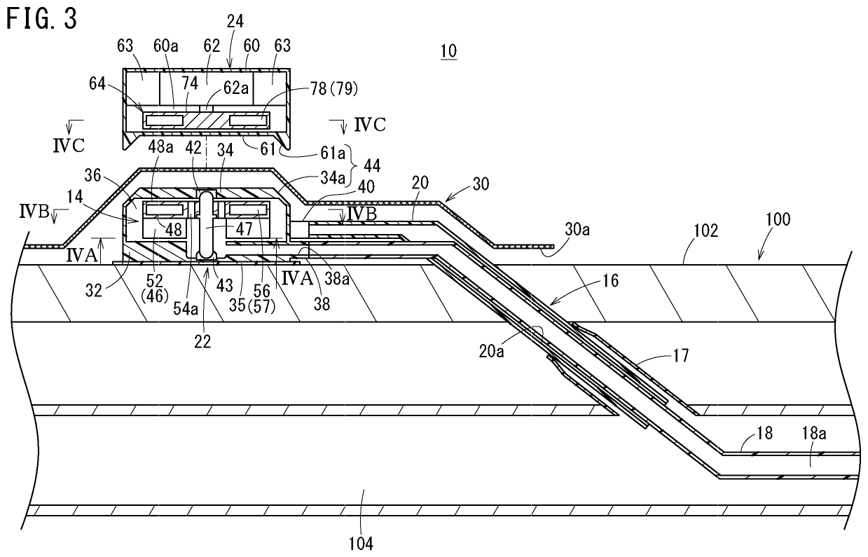

As shown in FIG. 1, an extracorporeal-circulation blood pump device 10 according to an embodiment of the present invention is used as an external ventricular assist device 12 that assists the cardiac function of a patient 100. The extracorporeal-circulation blood pump device 10 is configured as a centrifugal pump that includes an impeller 14 (see FIG. 3) in the device, and that removes or supplies blood of the patient 100 out of or into the body by the centrifugal force caused by the rotation of the impeller 14.

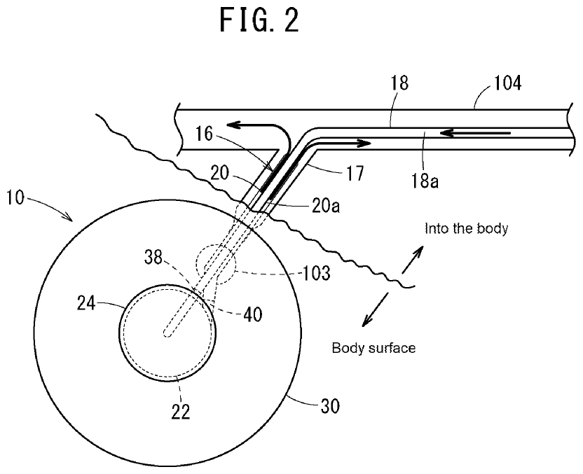

In the external ventricular assist device 12, a catheter 16 having a double tube structure is connected to the extracorporeal-circulation blood pump device 10 to form a circulation circuit that circulates blood between the patient 100 and the extracorporeal-circulation blood pump device 10 as shown in FIGS. 1 and 2. The catheter 16 includes an inner tube 18 (blood removal tube) having an inner lumen 18a, and an outer tube 20 (blood supply tube) having an outer lumen 20a that accommodates the inner tube 18. In the catheter 16, the inner tube 18 is exposed and separated from the outer tube 20 at the proximal end. The tubes 18 and 20 are connected to predetermined ports (blood inlet port 38 and blood outlet port 40) of the extracorporeal-circulation blood pump device 10.

For example, the catheter 16 is inserted into the subclavian artery 104 from a predetermined position on the body surface 102 of the patient 100 and further into the left ventricle 112 of the heart 110 via the aortic arch 106. While the catheter 16 is in place, a distal opening 18b of the inner tube 18 is placed in the left ventricle 112, and a distal opening 20b of the outer tube 20 is placed in the subclavian artery 104.

The external ventricular assist device 12 constructed as described above aspirates blood in the left ventricle 112 through the inner lumen 18a due to the centrifugal force of the extracorporeal-circulation blood pump device 10, and supplies the blood aspirated into the extracorporeal-circulation blood pump device 10 through the outer lumen 20a to the subclavian artery 104.

The extracorporeal-circulation blood pump device 10 according to the present embodiment is attached to the body surface 102 of the patient 100 and performs blood flow control while being carried by the patient 100.

Hereinafter, the extracorporeal-circulation blood pump device 10 will be described in detail.

As shown in FIGS. 1 to 3, the extracorporeal-circulation blood pump device 10 includes a pump body 22 that houses the impeller 14, a drive device 24 that rotates the impeller 14, a control unit 26 that controls driving of the drive device 24, and a battery 28 capable of supplying electric power. The extracorporeal-circulation blood pump device 10 includes a wound covering sheet 30 that covers an insertion location 103 of the catheter 16 and the pump body 22 together when being placed on the body surface 102 of the patient 100.

Further, the extracorporeal-circulation blood pump device 10 can transmit the driving force of the drive device 24 to the impeller 14 by assembling together the pump body 22 and the drive device 24 which are provided separately. After the extracorporeal-circulation blood pump device 10 is used, the pump body 22 is removed from the drive device 24 and discarded. In other words, the pump body 22 is configured to be a disposable type that is replaced for each use, and the used one is thrown away or sterilized. On the other hand, the drive device 24 is configured as a reuse type. Specifically, when the drive device 24 is used next time, a new pump body 22 is attached to the drive device 24, and the drive device 24 operates the impeller 14 of the newly attached pump body 22.

The pump body 22 of the extracorporeal-circulation blood pump device 10 includes a housing 32 formed to have an outer shape that can be mounted to the drive device 24. When being placed, the pump body 22 is attached to the body surface 102 while being placed on the body surface 102 side with respect to the drive device 24. The housing 32 has a cylindrical shape, and has flat wall parts 34 at its top and bottom. Inside the housing 32, the impeller 14 is housed in a rotatable manner, and an internal space 36 through which blood flows in and out is provided.

A pressure dispersion sheet 35 is attached to one of the wall parts 34 of the housing 32 (the wall part facing the body surface 102 when the pump body is placed). The pressure dispersion sheet 35 has a function of dispersing the contact pressure from the pump body 22 to the body surface 102. Examples of this type of sheet include those using polyisocyanate prepolymer gel, polyethylene gel, polyurethane foam, foamed silicone material, and the like. Further, a tapered part 34a inclined at a predetermined angle is provided on the outer peripheral edge of the other wall part 34 of the housing 32.

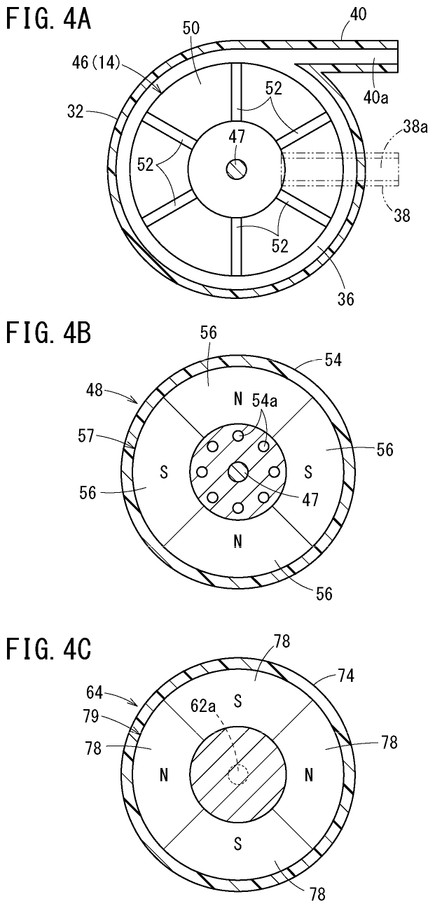

The blood inlet port 38 connected to the inner tube 18 of the catheter 16 and the blood outlet port 40 connected to the outer tube 20 of the catheter 16 are formed on the side peripheral surface of the housing 32. The blood inlet port 38 protrudes from the side surface of the housing 32, and has inside an inflow path 38a communicating with the internal space 36. The inflow path 38a extends in the housing 32 and is open near the center (a shaft member 47 described later) of the wall part 34 facing the body surface 102. The blood outlet port 40 protrudes in a tangential direction from the side surface of the housing 32, and has inside an outflow path 40a (see FIG. 4A) communicating with the internal space 36.

The impeller 14 accommodated in the internal space 36 generates a centrifugal force by rotation. The impeller 14 includes an upper fin section 46 and a lower driven rotating structure section 48 (rotor). Further, the impeller 14 has the shaft member 47 at the center of the driven rotating structure section 48. The shaft member 47 is supported to be rotatable by a first bearing 42 and a second bearing 43 provided in the housing 32. The first and second bearings 42 and 43 are formed as pivot bearings, for example.

The fin section 46 rotates together with the driven rotating structure section 48, thereby imparting a centrifugal force caused by the rotation to blood. For example, as shown in FIG. 4A, the fin section 46 includes a disk surface 50 extending radially outward from the shaft member 47 and a plurality of fins 52 protruding from the disk surface 50. The plurality of fins 52 is designed to have an inclination or a curved shape that generates an appropriate centrifugal force according to the rotation state (rotation direction, rotation speed, etc.) of the impeller 14.

The driven rotating structure section 48 constructs magnetic coupling that is magnetically coupled to the drive device 24. For example, the driven rotating structure section 48 is configured such that a plurality of driven magnets 56 (body-side magnetic coupling sections) is provided in a disk block 54 (see FIG. 4B) that extends radially outward from the shaft member 47. Further, the disk block 54 is formed with washout holes 54a penetrating the disk block 54 in the direction of the rotation axis at the inside of the driven magnets 56 and around the shaft member 47.

As shown in FIG. 4B, the plurality of driven magnets 56 is arranged along the circumferential direction at a predetermined distance from the central part (shaft member 47) to form an annular driven magnet group 57 (multipolar magnet group). In the driven magnet group 57, the polarities of the driven magnets 56 adjacent in the circumferential direction are different from each other. The material constituting the driven magnet 56 is not particularly limited, and examples of the driven magnet 56 include a permanent magnet such as an alnico magnet, a ferrite magnet, and a neodymium magnet. Instead of the driven magnet group 57, a single cylindrical magnet that is magnetized with multiple poles in the circumferential direction may be used as the driven magnet 56.

On the other hand, as shown in FIG. 3, the drive device 24 of the extracorporeal-circulation blood pump device 10 includes a casing 60, a motor mechanism 62 housed in the casing 60, a cooling mechanism 63 that cools the motor mechanism 62, and a drive rotating structure section 64 constituting magnetic coupling with the driven magnets 56 of the pump body 22.

The casing 60 is formed in a cylindrical shape having substantially the same outer diameter as the pump body 22. Inside the casing 60, a cavity 60a for accommodating the motor mechanism 62, the cooling mechanism 63, and the drive rotating structure section 64 is formed.

The casing 60 also has a recess 61 having a tapered outer peripheral part on an end wall where the drive rotating structure section 64 is disposed, and the pump body 22 is inserted and fitted such that the tapered part 34a of the pump body 22 is aligned with the tapered part 61a of the recess 61. That is, the recess 61 of the drive device 24 functions as an engagement mechanism 44 that positions and fixes the pump body 22 in a detachable manner.

As shown in FIGS. 2 and 3, the wound covering sheet 30 covers the treatment site of the patient 100 (the exposed portion of the catheter 16, the insertion location 103 of the catheter 16 into the body, etc.), in order to prevent invasion of bacteria and the like into the body. For the wound covering sheet 30 of this type, a material (for example, a film made of polyurethane, olefin, etc.) that permeates gas (water vapor, oxygen, etc.) and blocks liquid permeation is preferable, or non-woven fabric, vinyl chloride, cotton cloth, etc. can be applied.

An adhesive layer 30a that can be firmly adhered to the skin of a living body is provided on the surface of the wound covering sheet 30 on the body surface 102 side. For example, a gel adhesive, a hydrocolloid, or a silicone or acrylic adhesive may be applied to the adhesive layer 30a, and the adhesive layer 30a may contain a chemical solution such as a disinfectant.

In the wound covering sheet 30, the adhesive layer 30a is covered with a cover film (not shown) prior to the time that the wound covering sheet 30 is being attached to the patient 100, and when in use, the cover film is removed by a user. The cover film may have separate sections, wherein a portion on which the pump body 22 is to be mounted and a portion to be attached to the body surface 102 are separated from each other, and they may be removed independently.

The wound covering sheet 30 is formed to have a size sufficiently larger than the diameter of the casing 60. Thus, the wound covering sheet 30 can reliably cover the insertion location 103 where the catheter 16 is inserted into the body of the patient 100. The wound covering sheet 30 is not limited to be circular as long as it can cover and fix the pump body 22 and can cover the insertion location 103 of the catheter 16. For example, the wound covering sheet 30 may be formed into a polygonal shape which is long in the extending direction of the catheter 16.

The motor mechanism 62 of the drive device 24 can employ a well-known structure that includes a stator and a rotor (not shown) and that rotates a shaft 62a of the rotor. The motor mechanism 62 is electrically connected to the control unit 26, and the rotation speed of the shaft 62a is controlled by the control unit 26.

The cooling mechanism 63 cools the motor mechanism 62 when the motor mechanism 62 is driven, and has inside a pipe (not shown) through which a cooling medium such as water flows. The cooling medium cools the motor mechanism 62 by causing the cooling medium to flow under the control of the control unit 26 (depending on the driving and temperature of the motor mechanism 62). The cooling mechanism 63 may employ various configurations, and applicable examples of such configurations include an air cooling system and a heat dissipation structure. The cooling mechanism 63 may be mounted on the outside of the casing 60.

The drive rotating structure section 64 is configured such that a plurality of drive magnets 78 (drive-side magnetic coupling sections) is embedded in a holder 74 connected to the shaft 62a of the motor mechanism 62. As shown in FIG. 4C, the plurality of drive magnets 78 is arranged along the circumferential direction at the position facing the driven magnet group 57 to form an annular drive magnet group 79 (multipolar magnet group). In the drive magnet group 79, the polarities of the drive magnets 78 adjacent in the circumferential direction are different from each other. The material constituting the drive magnet 78 is also not particularly limited, and the materials described as the examples of the materials of the driven magnet 56 can be applied. Instead of the drive magnet group 79, a single cylindrical magnet that is magnetized with multiple poles in the circumferential direction may also be used as the drive magnet 78.

Returning to FIG. 1, the control unit 26 of the extracorporeal-circulation blood pump device 10 is composed of a known computer including an input/output interface, a memory, and a processor (not shown), and controls driving of the motor mechanism 62. A monitor, operation buttons, and the like (not shown) are provided on the outer surface of the control unit 26, and the user sets the details of the driving of the extracorporeal-circulation blood pump device 10 by operating the operation buttons. The control unit 26 controls the power supply of the battery 28 on the basis of information set by the user, and rotates the shaft 62a in a range of, for example, 0 to 30000 rpm.

The control unit 26 and the battery 28 are attached to a band 29 that can be wound around the body of the patient 100. Thus, the patient 100 can easily carry the external ventricular assist device 12 including the extracorporeal-circulation blood pump device 10 and can perform daily activities. The control unit 26 and the battery 28 may be stored together in a storage bag 25 (see the dotted line in FIG. 1).

The extracorporeal-circulation blood pump device 10 according to the present embodiment is basically configured as described above, and the operation and effect thereof will be described below.

As shown in FIG. 1, the external ventricular assist device 12 including the extracorporeal-circulation blood pump device 10 is constructed for the patient 100 requiring support for the cardiac function. A user such as a doctor inserts the catheter 16 into the body from a predetermined location on the body surface 102 of the patient 100 (insertion location 103 near the upper part of the right breast). This catheter 16 reaches the left ventricle 112 of the heart 110 through the subclavian artery 104. The distal opening 18b of the inner tube 18 is placed in the left ventricle 112, and the distal opening 20b of the outer tube 20 is placed in the subclavian artery 104. An artificial blood vessel 17 (see FIGS. 2 and 3) that externally encloses the catheter 16 and guides insertion of the catheter 16 may be disposed in a region from the insertion location 103 of the catheter 16 to the subclavian artery 104.

The user connects the inner tube 18 at the proximal end of the catheter 16 exposed on the body surface 102 to the blood inlet port 38 of the pump body 22, and connects the outer tube 20 at the proximal end to the blood outlet port 40 of the pump body 22. When constructing the external ventricular assist device 12, the user assembles the extracorporeal-circulation blood pump device 10 by mounting the drive device 24 to the pump body 22 as shown in FIG. 3. During the assembly, the wound covering sheet 30 is attached to the body surface 102 by the user with the adhesive layer 30a facing the body surface 102 (the pump body 22 and the catheter 16). Further, the wound covering sheet 30 is affixed to the insertion location 103 (wound) of the catheter 16 to cover the part of the catheter 16 exposed on the body surface and the pump body 22 together.

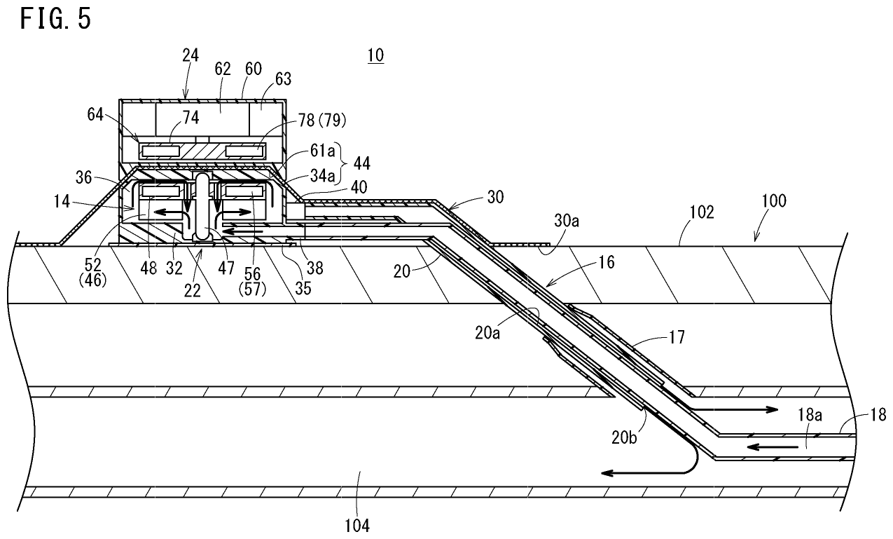

In a state where the part of the catheter 16 exposed on the body surface and the pump body 22 are affixed to the body surface 102 by the wound covering sheet 30, the drive device 24 is mounted to the pump body 22 such that the tapered part 34a fits the recess 61 (tapered part 61a). As a result, as shown in FIG. 5, the driven magnet group 57 in the pump body 22 and the drive magnet group 79 in the drive device 24 are magnetically coupled, and the drive device 24 fitted to the pump body 22 is disposed on the outside of the wound covering sheet 30. The drive device 24 is electrically connected to the control unit 26.

The problems such as inadvertent detachment or displacement of the pump body 22 and the catheter 16 covered with the wound covering sheet 30 during activities of the patient 100 are less likely to occur. In particular, although the pump body 22 and the drive device 24 are likely to be displaced because they have a certain amount of weight, the wound covering sheet 30 places the pump body 22 and the drive device 24 at the center and firmly fixes the region around them, thereby being capable of holding them on the body surface 102 in a stable manner. The drive device 24 may be fixed to the patient 100 by a stretchable bra-like undergarment or a harness.

When the extracorporeal-circulation blood pump device 10 is assembled, the drive magnet group 79 of the drive device 24 and the driven magnet group 57 of the pump body 22 form magnetic coupling. The drive device 24 rotates the drive magnet group 79 at an arbitrary rotation speed under the control of the control unit 26. In the extracorporeal-circulation blood pump device 10 placed on the body surface 102, the driven magnet group 57 is rotated along with the rotation of the drive magnet group 79 to rotate the impeller 14 in the pump body 22.

The fin section 46 of the impeller 14 generates a centrifugal force as it rotates, allowing blood to flow into the internal space 36 via the inner tube 18 and the blood inlet port 38, and allowing blood to flow out of the internal space 36 via the blood outlet port 40 and the outer tube 20. Thus, the external ventricular assist device 12 can satisfactorily circulate (remove or supply) the blood of the patient 100.

A portion of the blood flowing through the blood inlet port 38 by the fin section 46 flows toward the back surface 48a of the driven rotating structure section 48, and again flows into the fin section 46 through the washout holes 54a provided around the shaft member 47. Such a structure can prevent blood from staying and coagulating on the back surface 48a of the driven rotating structure section 48.

As described above, the extracorporeal-circulation blood pump device 10 according to the present embodiment is stably and hygienically placed on the body surface 102 of the patient 100 with a simple configuration having the wound covering sheet 30. In particular, when attached, the wound covering sheet 30 extends radially outward with respect to the pump body 22 and covers the insertion location 103 of the catheter 16 and the pump body 22 together at once. Thus, this configuration can reliably prevent the positional displacement of the extracorporeal-circulation blood pump device 10 and the catheter 16 by preventing the portion where the blood flows from being exposed, and can satisfactorily block invasion of bacteria into the body.

The extracorporeal-circulation blood pump device 10 includes the cooling mechanism 63, so that the motor mechanism 62 of the extracorporeal-circulation blood pump device 10 placed on the body surface 102 of the patient 100 can be easily cooled while being driven. In addition, the pressure dispersion sheet 35 of the extracorporeal-circulation blood pump device 10 can suppress the extracorporeal-circulation blood pump device 10 from pressing the body surface 102 at the location where the sheet is attached.

Furthermore, the double-tube catheter 16 of the external ventricular assist device 12 is inserted into one blood vessel (subclavian artery 104) from the body surface 102 of the patient 100, whereby the burden on the patient 100 can be reduced. In particular, the wound covering sheet 30 only needs to cover one location where the catheter 16 is inserted together with the pump body 22, whereby the wound covering sheet 30 can be more easily attached to the patient 100.

Several modifications of the extracorporeal-circulation blood pump device according to the present invention will be described below. In the following description, components having the same functions as those of the extracorporeal-circulation blood pump device 10 are given the same reference numerals, and detailed descriptions thereof are omitted.

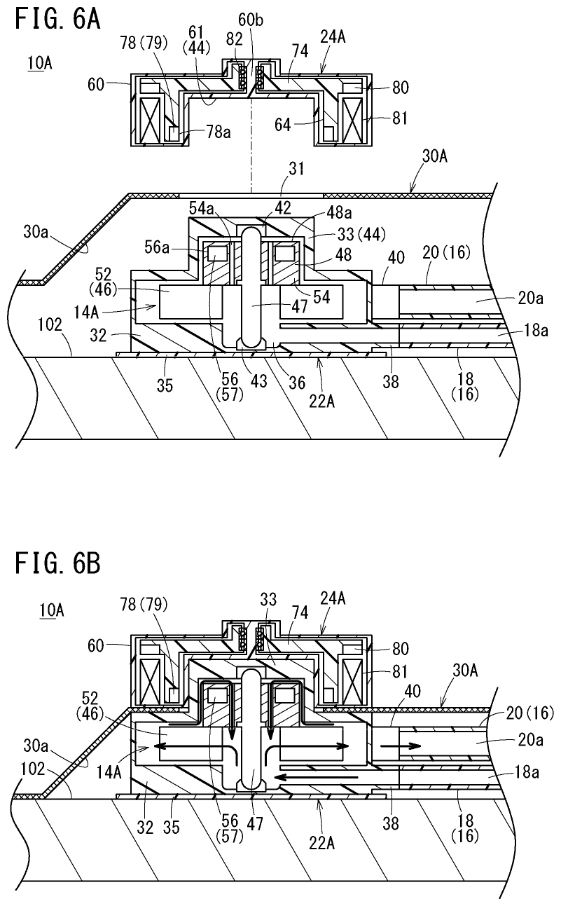

An extracorporeal-circulation blood pump device 10A according to the first modification shown in FIGS. 6A and 6B is different from the extracorporeal-circulation blood pump device 10 according to the present embodiment in that a wound covering sheet 30A has an opening 31, and a driven rotating structure section 48 of a pump body 22A is disposed in the opening 31 of the wound covering sheet 30A. Further, the wound covering sheet 30A is fixed to the pump body 22A with an adhesive or the like (adhesive layer 30a), and a drive rotating structure section 64 of a drive device 24A is mounted to the pump body 22A exposed from the opening 31 of the wound covering sheet 30A in a detachable manner.

The extracorporeal-circulation blood pump device 10A is also different in that a driven magnet 56 in an impeller 14A and a drive magnet 78 in the drive device 24A form magnetic coupling in the radial direction, not in the axial direction. The driven rotating structure section 48 of the pump body 22A is disposed on a cylindrical protruding part 33 of a housing 32 with a magnetic coupling surface 56a of the driven magnet 56 being directed to the outer periphery.

The opening 31 of the wound covering sheet 30A is formed to have a diameter slightly larger than the diameter of the protruding part 33 of the housing 32. Therefore, the protruding part 33 of the housing 32 can be easily exposed to the outside from the opening 31 of the wound covering sheet 30. The wound covering sheet 30A is attached to the body surface 102 so as to cover the pump body 22A and a part of the catheter 16 exposed on the body surface together while affixing and holding the pump body 22A.

A casing 60 of the drive device 24A is formed in a bottomed cylindrical shape as a whole, and has a recess 61 into which the protruding part 33 of the pump body 22A can be fitted. Further, the drive device 24A includes inside the drive magnet 78 having a magnetic coupling surface 78a directed to the inner periphery of the recess 61, a motor rotor 80 disposed on a holder 74 that holds the drive magnet 78, and a stator 81 that exerts a rotating magnetic field on the motor rotor 80. The holder 74 that holds the drive magnet 78 and the motor rotor 80 is pivotally supported by a bearing 82 on a shaft support part 60b of the casing 60 so as to be relatively rotatable.

When the protruding part 33 of the pump body 22A is fitted to the recess 61 of the drive device 24A, the drive magnet 78 in the drive device 24A and the driven magnet 56 in the pump body 22A form magnetic coupling. Therefore, when the motor rotor 80 in the drive device 24A rotates, the drive magnet 78 rotates, and the driven magnet 56 rotates along with the rotation of the drive magnet 78. A fin 52 integrally formed with the driven rotating structure section 48 rotates, so that blood flowing in through the blood inlet port 38 is sent to the blood outlet port 40.

Therefore, the extracorporeal-circulation blood pump device 10A according to the first modification can also provide the same effects as those of the extracorporeal-circulation blood pump device 10 according to the present embodiment. In particular, the extracorporeal-circulation blood pump device 10A is configured such that, due to the opening 31 formed in the wound covering sheet 30A, the pump body 22A is exposed and mounted to the drive device 24A in direct contact therewith in a detachable manner. The pump body 22A and the drive device 24A can be assembled together with the wound covering sheet 30A interposed therebetween. As a result, the wound covering sheet 30A can easily affix the pump body 22A onto the body surface 102. Further, the positional displacement between the pump body 22A and the drive device 24A can be prevented more reliably, and thus, the positional relationship between the driven magnet 56 and the drive magnet 78 can be more accurately fixed, which enables stable torque transmission. That is, the extracorporeal-circulation blood pump device 10A is placed on the patient 100 more stably and hygienically. In the present modification, the magnetization direction (magnetic coupling direction) of the driven magnet 56 and the drive magnet 78 coincides with the radial direction. However, the magnetization direction may coincide with the axial direction, or the magnetization direction may be inclined with respect to the rotation axis of the impeller 14A.

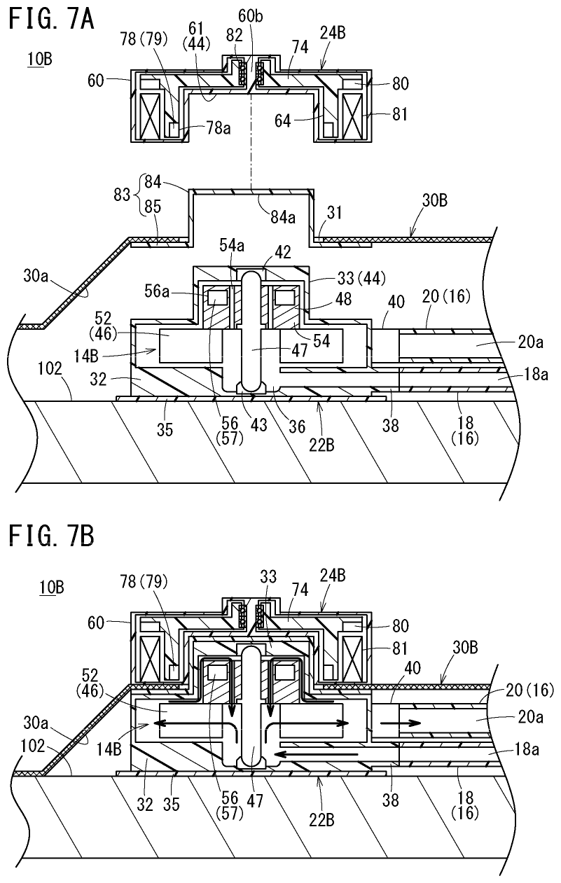

An extracorporeal-circulation blood pump device 10B according to the second modification shown in FIGS. 7A and 7B is different from the extracorporeal-circulation blood pump device 10A according to the first modification in that a hard cover 83 airtightly integrated with a wound covering sheet 30B is disposed in the opening 31 shown in the first modification.

Specifically, the wound covering sheet 30B is provided with an opening 31 having a diameter slightly larger than the diameter of the protruding part 33 of the housing 32, and the hard cover 83 is mounted to penetrate the wound covering sheet 30B through the opening 31. The hard cover 83 is made of a hard material such as polycarbonate, acryl, or polysulfone.

Further, in a side sectional view, the hard cover 83 has a protruding shape having a projecting part 84 exposed from the wound covering sheet 30B and a flange part 85 which protrudes radially outward from the projecting part 84 and on which the wound covering sheet 30B is placed on the opposite side from the body surface 102. A cylindrical recess 84a capable of accommodating the cylindrical protruding part 33 of the pump body 22B is provided in the projecting part 84 on the side facing the body surface 102. The hard cover 83 and the wound covering sheet 30B are integrated with each other in advance in an airtight manner, and the integrated wound covering sheet 30B and the hard cover 83 are attached to the body surface 102 so as to cover the pump body 22B and the part of the catheter 16 exposed on the body surface together.

The pump body 22B is configured in the same manner as the pump body 22A in the first modification, and a driven rotating structure section 48 of an impeller 14B is disposed in the protruding part 33 with a magnetic coupling surface 56a of a driven magnet 56 being directed to the outer periphery. The drive device 24B is also configured in the same manner as the drive device 24A according to the first modification, and has a recess 61 that can accommodate the protruding part 33 of the pump body 22B with the hard cover 83 interposed therebetween. The drive device 24B includes inside a drive magnet 78 having a magnetic coupling surface 78a directed to the inner periphery of the recess 61, a motor rotor 80 disposed on a holder 74 that holds the drive magnet 78, and a stator 81 that exerts a rotating magnetic field on the motor rotor 80. The holder 74 that holds the drive magnet 78 and the motor rotor 80 is rotatable by a bearing 82.

The projecting part 84 of the hard cover 83 having the cylindrical recess 84a is concentrically formed so that the recess 61 of the drive device 24B can be fitted thereto. When the projecting part 84 of the hard cover 83 is fitted to the recess 61 of the drive device 24B, the drive magnet 78 in the drive device 24B and the driven magnet 56 in the protruding part 33 fitted into the cylindrical recess 84a form magnetic coupling. That is, when the motor rotor 80 in the drive device 24B rotates, the drive magnet 78 rotates, and the driven magnet 56 rotates along with the rotation of the drive magnet 78. As a result, a fin 52 integrally formed with the driven rotating structure section 48 rotates, so that blood flowing in through the blood inlet port 38 is sent to the blood outlet port 40.

In particular, the extracorporeal-circulation blood pump device 10B is configured such that the drive device 24B is mounted to the pump body 22B in a detachable manner with the hard cover 83 airtightly integrated with the wound covering sheet 30B interposed therebetween, whereby the pump body 22B can be more reliably sealed from the outside. Further, it is possible to reliably prevent the positional displacement between the pump body 22B and the drive device 24B. Furthermore, if the wound covering sheet 30B and the extracorporeal-circulation blood pump device 10B are not fixed with an adhesive or the like, the wound covering sheet 30B can be more easily separated from blood pump device 10B in order to replace wound covering sheet 30B.

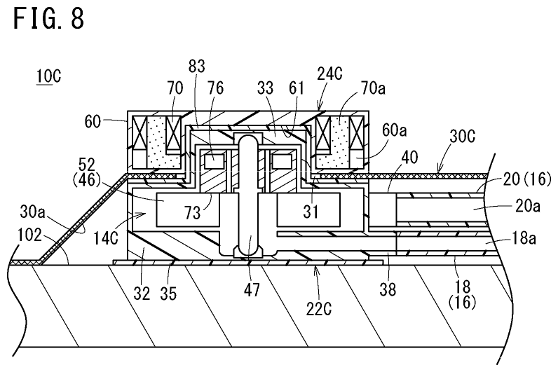

An extracorporeal-circulation blood pump device 10C according to the third modification shown in FIG. 8 is different from the extracorporeal-circulation blood pump device 10B according to the second modification in that an impeller 14C housed in a pump body 22C functions as a rotor, and only a stator 70 is provided inside a drive device 24C.

The impeller 14C has a rotor section 73 in place of the driven rotating structure section 48 on the side opposite to the fin section 46. The rotor section 73 is rotatable around the axis of the shaft member 47, and is provided with a plurality of working magnets 76 (permanent magnets) magnetized in the radial direction. The working magnets 76 rotate the impeller 14C in a predetermined direction by receiving an acting force based on the magnetization of the stator 70 when the extracorporeal-circulation blood pump device 10C is driven. Further, the housing 32 of the pump body 22C has a protruding part 33 that can accommodate the rotor section 73 of the impeller 14C inside.

On the other hand, the casing 60 of the drive device 24C has a recess 61 into which the protruding part 33 can be inserted and fitted, and includes a stator 70 in a cavity 60a outside the recess 61 in the width direction. The stator 70 of the drive device 24C has an iron core 70a extending in a predetermined direction and facing the working magnets 76. The stator 70 is connected to the control unit 26 (see FIG. 1), and rotates the rotor section 73 of the impeller 14C at an appropriate rotation speed under the control of the control unit 26.

Further, a wound covering sheet 30C similar to the wound covering sheet 30B according to the second modification is provided between the pump body 22C and the drive device 24C. That is, the wound covering sheet 30C is integrally formed with a hard cover 83 which is inserted into the opening 31, and covers the pump body 22C exposed on the body surface 102 and the exposed part of the catheter 16 together.

Therefore, the extracorporeal-circulation blood pump device 100 according to the third modification can also provide the same effects as those of the extracorporeal-circulation blood pump devices 10, 10A, and 10B according to the present embodiment. In particular, the extracorporeal-circulation blood pump device 100 constitutes a motor mechanism that directly rotates the impeller 14C by detachably mounting the pump body 22C to the drive device 24C. Since there is no movable section (rotor section) in the drive device 24C, it is possible to further improve the mechanical durability and reliability of the extracorporeal-circulation blood pump device 100.

The drive device 24C can be configured to include a sensor (not shown) for detecting the radial position of the rotor section 73 and to include a winding for controlling the radial position of the rotor section 73 on the stator 70, as disclosed in U.S. Pat. No. 6,053,705. Thus, a bearingless motor can be constituted by the rotor section 73 and the stator 70. Accordingly, the extracorporeal-circulation blood pump device 100 can also be configured such that the impeller 14C floats and rotates in the housing 32 without having the shaft member 47 of the impeller 14C and the first and second bearings 42 and 43.

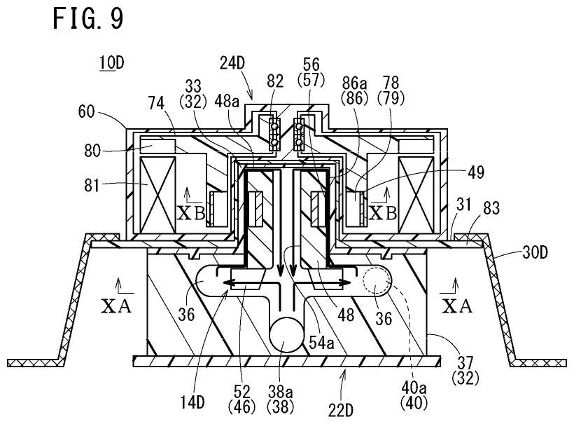

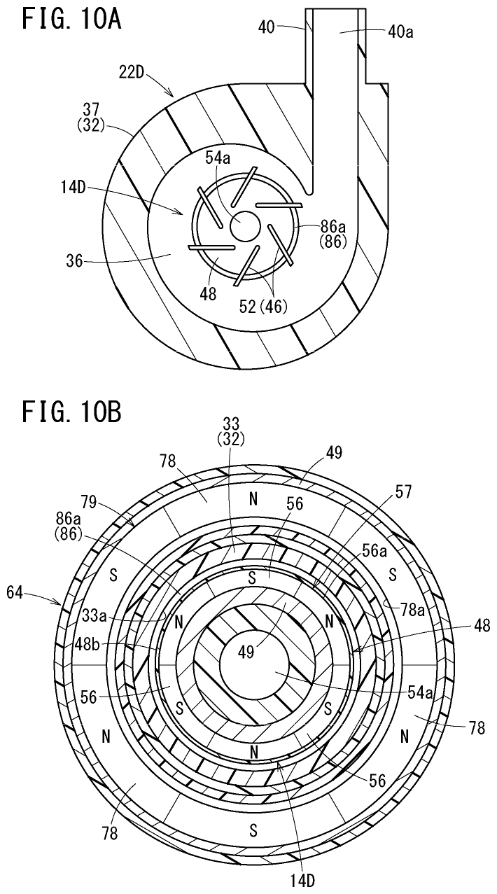

An extracorporeal-circulation blood pump device 10D according to the fourth modification shown in FIGS. 9, 10A, and 10B is different from the extracorporeal-circulation blood pump devices 10 and 10A to 10C in that a dynamic pressure bearing 86 is constituted by the outer periphery of a driven rotating structure section 48 and the inner periphery of a housing 32 that houses the driven rotating structure section 48. The driven rotating structure section 48 is supported so as to float and rotate in the radial direction of an impeller 14D by the dynamic pressure bearing 86. Further, the driven rotating structure section 48 is held at a substantially fixed position in the direction of the rotation axis by a passive magnetic bearing constituted by a driven magnet 56 (magnetic body) in the driven rotating structure section 48 and a drive magnet 78 (magnetic body) in a drive device 24D. Further, a back yoke 49 for suppressing magnetic leakage is provided on the surface reverse to the magnetic coupling surface 56a of the driven magnet 56 and the surface reverse to the magnetic coupling surface 78a of the drive magnet 78.

The extracorporeal-circulation blood pump device 10D is different from the extracorporeal-circulation blood pump device 10B according to the second modification only in the method for supporting and rotating the impeller 14D in the pump body 22D, and is the same in other configurations (drive device 24D and wound covering sheet 30D). That is, the wound covering sheet 30D has an opening 31 in which the hard cover 83 is disposed to pass therethrough, and the hard cover 83 is sandwiched between the pump body 22D and the drive device 24D. Note that the wound covering sheet 30D is fixed to the hard cover 83 on the radially outer side of the drive device 24D.

The impeller 14D in the extracorporeal-circulation blood pump device 10D includes the driven rotating structure section 48 that houses the driven magnet 56 magnetized in the circumferential direction, and a fin 52 (fin section 46) formed integrally with the driven rotating structure section 48. The driven rotating structure section 48 is disposed in a cylindrical protruding part 33 of the housing 32, and the fin 52 is accommodated in a large-diameter part 37 of the housing 32. An internal space 36 formed in the large-diameter part 37 is formed to have a substantially cross-shaped cross section in a side view. An inflow path 38a communicates with the central part of the large-diameter part 37, and an outflow path 40a communicates with the outer periphery of the space which is wide in the radial direction.

A clearance, which forms the dynamic pressure bearing 86, between the inner peripheral surface 33a of the protruding part 33 and the outer peripheral surface 48b of the driven rotating structure section 48 is formed to be sufficiently narrow so as to act as a dynamic pressure bearing clearance 86a. The size of the dynamic pressure bearing clearance 86a is preferably 0.2 mm or less. Due to the dynamic pressure generated in the dynamic pressure bearing clearance 86a during rotation, the impeller 14D can float and rotate in the radial direction. As described above, the position of the impeller 14D in the direction of the rotation axis with respect to the housing 32 is controlled so that the driven magnet 56 is located on the substantially same position as the drive magnet 78 in the direction of the rotation axis by the action of the passive magnetic bearing due to the attractive force between the driven magnet 56 and the drive magnet 78.

In the extracorporeal-circulation blood pump device 10D configured as described above, a portion of blood flowing through the blood inlet port 38 by the fin section 46 flows toward the back surface 48a of the driven rotating structure section 48, and again flows into the fin section 46 through a washout hole 54a provided in the center of the driven rotating structure section 48. Such a structure can prevent blood from staying and coagulating on the back surface 48a of the driven rotating structure section 48.

The impeller 14D of the extracorporeal-circulation blood pump device 10D can rotate, while floating, without contacting the housing 32, thereby being capable of circulating blood more safely by preventing blood destruction or coagulation due to friction or the like. Further, since the shaft member 47 and the storage space therefor are not required, the pump body 22D can be made more compact, and the manufacturing cost can be reduced.

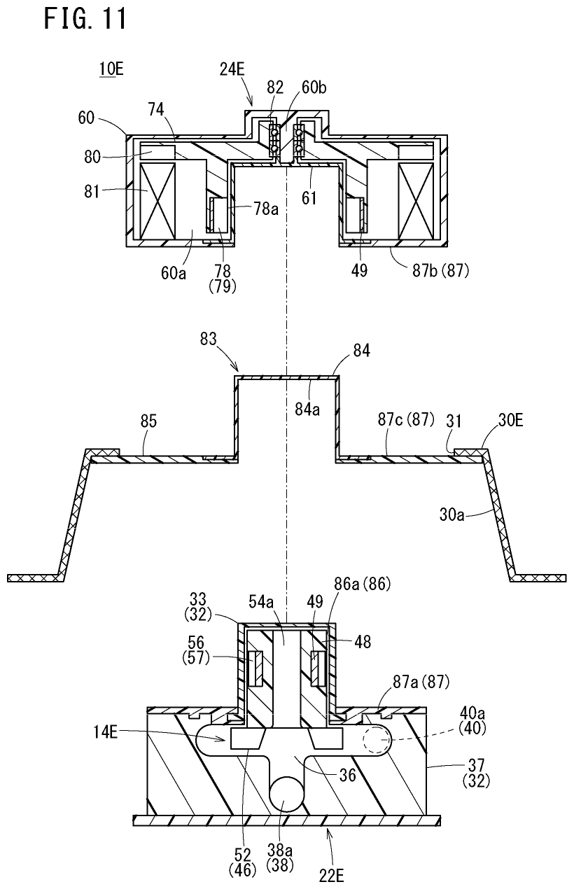

An extracorporeal-circulation blood pump device 10E according to the fifth modification shown in FIG. 11 is different from the extracorporeal-circulation blood pump devices 10 and 10A to 10D in that at least a part of a housing 32 of a pump body 22E and at least a part of a casing 60 of a drive device 24E are made of a thermal conductive member 87. Furthermore, in this modification, the thermal conductive member 87 is also provided on a part of a hard cover 83 to which a wound covering sheet 30E is fixed. The configurations other than the thermal conductive member 87 are the same as those of the extracorporeal-circulation blood pump device 10D according to the fourth modification.

The thermal conductive member 87 is formed of a material having high thermal conductivity, and examples of such material include metal such as aluminum, a ceramic such as alumina, and a carbon fiber reinforced resin. Specifically, the housing 32 of the pump body 22E has a thermal conductive member 87a at a portion facing the drive device 24E (hard cover 83) excluding the protruding part 33. A part of the thermal conductive member 87a reaches an inner wall constituting an internal space 36 from an exposed portion of the upper surface of the housing 32. On the other hand, the casing 60 of the drive device 24E is enclosed by a thermal conductive member 87b on the entire outer surface of the casing 60 except for a recess 61, and a shaft support part 60b that supports a holder 74 is also formed from the thermal conductive member 87b. On the other hand, the hard cover 83 has a projecting part 84 and a flange part 85, and most of the flange part 85 other than the area connected to the projecting part 84 is formed from a thermal conductive member 87c.

In the extracorporeal-circulation blood pump device 10E configured as described above, the thermal conductive members 87a, 87b, and 87c are laminated on each other when the pump body 22E, the drive device 24E, and the wound covering sheet 30E (including the hard cover 83) are assembled together. Therefore, heat generated by driving a motor of the drive device 24E is transmitted to the thermal conductive member 87a of the pump body 22E from the thermal conductive member 87b via the thermal conductive member 87c of the hard cover 83. The thermal conductive member 87a transfers heat to the internal space 36, and the transferred heat moves into blood. Therefore, the motor mechanism that rises in temperature when driven can be easily cooled. Further, the impeller 14E rotates with the rotation of the motor mechanism, and allows blood to flow into the internal space 36 through the blood inlet port 38 and discharges blood through the blood outlet port 40 by the fin section 46.

In the extracorporeal-circulation blood pump device 10E, the hard cover 83 is integrated with the opening 31 of the wound covering sheet 30E. However, the hard cover 83 may not be provided in some embodiments, and in this case, the portion of the pump body 22E formed from a material having high thermal conductivity and the portion of the drive device 24E formed from a material having high thermal conductivity may be in direct contact with each other in the opening 31 of the wound covering sheet 30E. Further, in the extracorporeal-circulation blood pump device 10E, the protruding part 33 of the housing 32 and the recess 61 in the casing 60, which are disposed between the driven magnet 56 in the pump body 22E and the drive magnet 78 in the drive device 24E, are preferably formed from a resin such as polycarbonate in order to prevent generation of eddy currents and to reduce manufacturing cost. However, when more heat transfer areas are required, such portions may also be formed from a material having high thermal conductivity. In this case, ceramic that does not generate eddy current is most preferable, and the next preferable material is carbon fiber reinforced resin that generates less eddy current than metal.

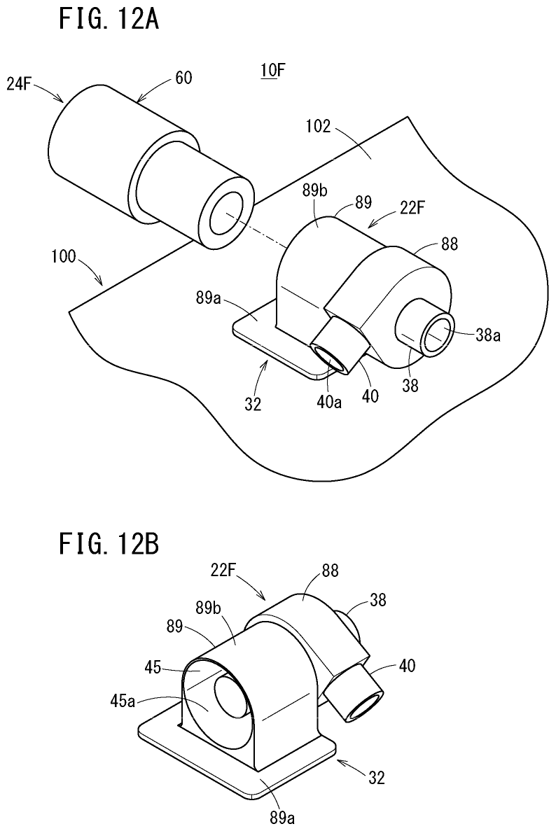

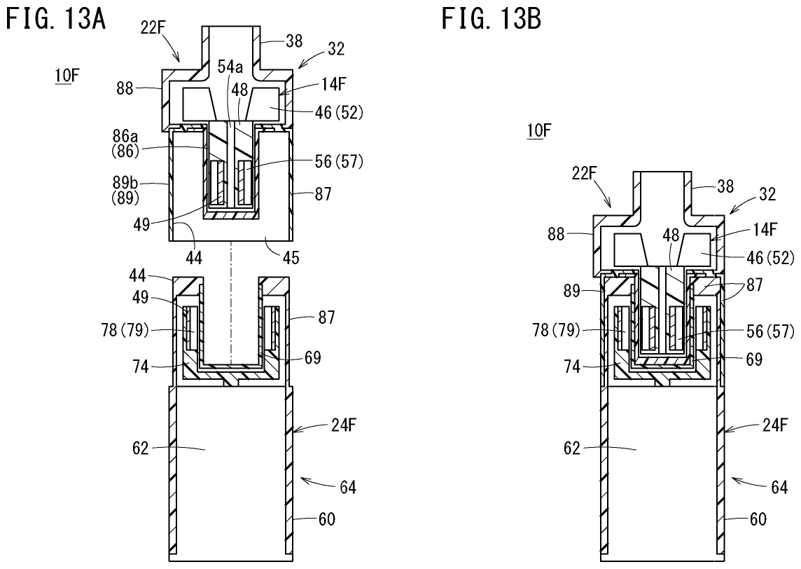

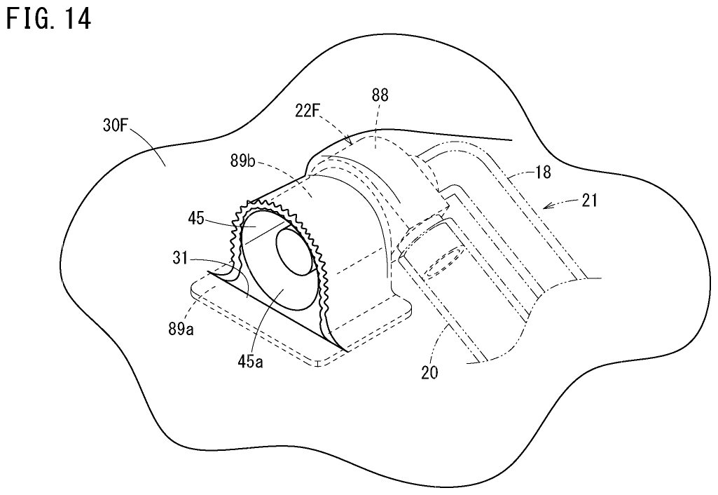

An extracorporeal-circulation blood pump device 10F according to the sixth modification shown in FIGS. 12A to 14 is different from the extracorporeal-circulation blood pump devices 10 and 10A to 10E in that the extracorporeal-circulation blood pump device 10F has a pump body 22F disposed such that the rotation axis of an impeller 14F is parallel to the body surface 102. Further, a drive rotating structure section 64 in a drive device 24F has a motor mechanism 62 having a rotation axis coaxial with the rotation axis of the impeller 14F, and a drive magnet 78 is attached to a holder 74 of the motor mechanism 62.

A wound covering sheet 30F has a slit-shaped opening 31, and an adhesive layer 30a made of an adhesive or the like is attached to the pump body 22F. When the wound covering sheet 30F is attached to the pump body 22F, a connection part (one of engagement mechanisms 44) of the pump body 22F is disposed in the opening 31 of the wound covering sheet 30F, and is exposed through the opening 31, thereby being capable of being engaged with a connection part (the other of the engagement mechanisms 44) of the drive device 24F in a detachable manner.

A housing 32 of the pump body 22F has a first portion 88 where a fin section 46 is disposed and a second portion 89 where a driven rotating structure section 48 is disposed. The first portion 88 includes a blood inlet port 38 and a blood outlet port 40 connected to an inflow/outflow cannula 21 inserted into a blood vessel in the body. The second portion 89 is formed into a semi-cylindrical shape connected to a rectangular affixing base 89a and rises from the affixing base 89a in plan view. An insertion hole 45 into which the drive device 24F is inserted and fitted is formed in a raised part 89b of the second portion 89. The affixing base 89a is placed (attached) so as to be in surface contact with the body surface 102, thereby holding the rotation axis of the impeller 14F of the pump body 22F in parallel.

The raised part 89b of the pump body 22F widens the slit-shaped opening 31 in the wound covering sheet 30F, and exposes the insertion hole 45 outside the wound covering sheet 30F. The wound covering sheet 30F is attached to the body surface 102 so as to cover the second portion 89 (excluding the insertion hole 45), the first portion 88, and a part of the inflow/outflow cannula 21 exposed on the body surface together.

When being used, the extracorporeal-circulation blood pump device 10F is assembled in such a manner that the drive device 24F is inserted into the insertion hole 45 exposed from the opening 31 of the wound covering sheet 30F. Thus, the driven magnet 56 in the driven rotating structure section 48 of the pump body 22F and the drive magnet 78 in the drive device 24F constitute magnetic coupling along the radial direction of the rotation axis. Therefore, due to the rotation of the motor mechanism 62 in the drive device 24F, the drive magnet 78 rotates, and the driven magnet 56 rotates with the rotation of the drive magnet 78. Thus, the fin 52 connected to the impeller 14F rotates, whereby blood flowing in through the blood inlet port 38 is sent through the blood outlet port 40. A clearance between the outer periphery of the driven rotating structure section 48 and the inner periphery of the housing 32 is formed as a dynamic pressure bearing clearance 86a, and the impeller 14F is supported so as to float and rotate in the radial direction by the dynamic pressure.

The impeller 14F is held at a substantially fixed position in the direction of the rotation axis by a passive magnetic bearing constituted by the driven magnet 56 in the driven rotating structure section 48 and the drive magnet 78 in the drive device 24F. Further, the outer periphery 45a constituting the insertion hole 45 of the housing 32 of the pump body 22F and a part of the outer periphery of the drive device 24F are both formed from a thermal conductive member 87 having high thermal conductivity such as metal, ceramic, or carbon fiber reinforced resin, and they are at least partially in contact with each other when the drive device 24F is inserted into the insertion hole 45 of the pump body 22F. An inner periphery 69 of the drive device 24F is formed as a hard wall part that can be firmly fitted to the pump body 22. With such a configuration, heat generated in the drive device 24F is radiated to the blood flowing in the pump body 22F via the thermal conductive member 87, so that the drive device 24F is cooled.

In the extracorporeal-circulation blood pump device 10F configured as described above, the impeller 14F which is long in the direction of the rotation axis and has a small diameter rotates at a higher speed. Therefore, the pump body 22F can be placed on the body surface 102 without increasing the height of the pump body 22F protruding from the body surface 102.

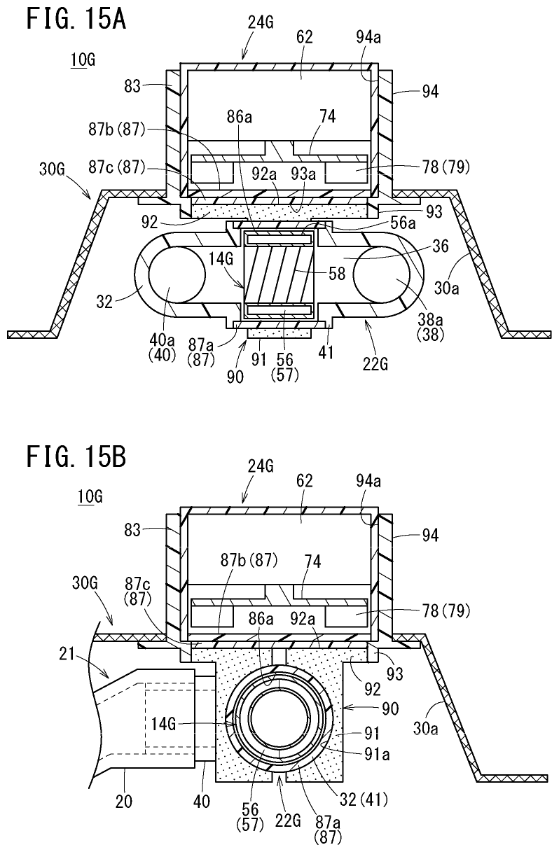

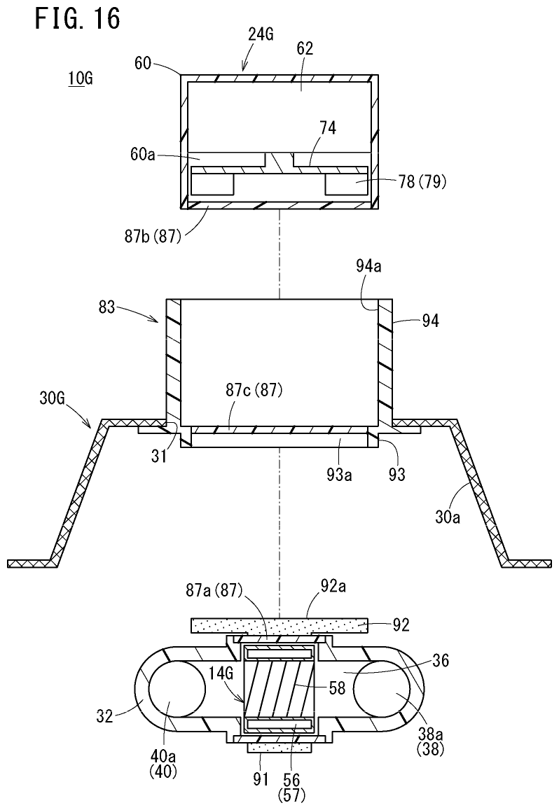

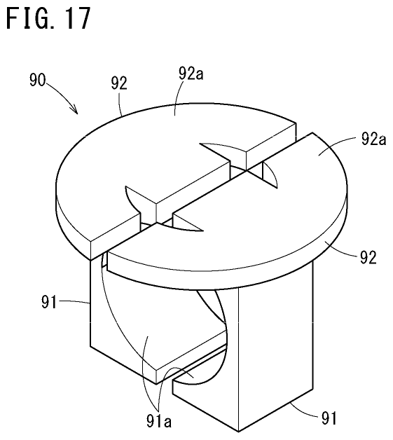

An extracorporeal-circulation blood pump device 10G according to the seventh modification shown in FIGS. 15A to 17 is different from the extracorporeal-circulation blood pump devices 10 and 10A to 10F which are of a centrifugal type in that the extracorporeal-circulation blood pump device 10G is of an axial flow type. The extracorporeal-circulation blood pump device 10G is configured so that a rotation axis of an impeller 14G in a pump body 22G is parallel to the body surface 102, as in the extracorporeal-circulation blood pump device 10F according to the sixth modification. Furthermore, a drive device 24G includes a motor mechanism 62 that rotates a holder 74 around a rotation axis perpendicular to the rotation axis of the impeller 14G, and a drive magnet 78 is fixed to the holder 74. A magnetic body (driven magnet 56) in the impeller 14G and the drive magnet 78 in the drive device 24G are magnetically coupled via a pole piece 90 made of a soft magnetic material.

A housing 32 of the pump body 22G is provided with a blood inlet port 38 and a blood outlet port 40 connected to an inflow/outflow cannula 21 inserted into a blood vessel in the body, and is formed in a cylindrical shape extending between these ports. The pump body 22G is disposed so that the cylindrical axis (the rotation axis of the impeller 14G) is substantially parallel to the body surface 102.

A large part 41 that is larger in the radial direction than the connection portions with the ports 38 and 40 is formed in a substantially central part of the cylindrical housing 32, and the cylindrical impeller 14G is disposed inside the large part 41. A driven magnet 56 magnetized with two magnetic poles along the circumferential direction and having a magnetic coupling surface 56a on the outer peripheral surface is embedded in the cylinder of the impeller 14G. The impeller 14G has an axial flow fin 58 on its inner peripheral surface.

A pair of pole pieces 90 made of a soft magnetic material is disposed on the outer periphery of the large part 41 of the housing 32. Each of the pair of pole pieces 90 includes a body-side mounting part 91 that is mounted on the housing 32 of the pump body 22G, and a drive-side mounting part 92 that is connected to one end of the body-side mounting part 91 and faces the drive device 24G. The pair of pole pieces 90 is formed symmetrical with respect to the housing 32.

The pair of body-side mounting parts 91 has a rectangular outer shape as a whole, and has magnetic coupling surfaces 91a on which the pump body 22G is placed at portions facing each other on the inner side. The magnetic coupling surfaces 91a are formed into a semicircular shape and magnetically coupled to the driven magnet 56 in the impeller 14G. The pair of drive-side mounting parts 92 is disposed on the opposite side from the body surface 102, and has substantially semi-circular magnetic coupling surfaces 92a magnetically coupled to the drive magnet 78 magnetized with two magnetic poles in the drive device 24G. The magnetic coupling surfaces 92a are disposed so as to be substantially parallel to the rotation axis of the impeller 14G. Therefore, the center axis of the magnetic coupling surfaces 92a of the pole pieces 90 on the drive device 24G side is perpendicular to the rotation axis of the impeller 14G when the pump body 22G, the drive device 24G, and the pole pieces 90 are assembled together.

On the other hand, a wound covering sheet 30G is provided with an opening 31, and a hard cover 83 is attached around the opening 31. The hard cover 83 includes a lower cylinder part 93 having a cylindrical recess 93a capable of housing the magnetic coupling surfaces 92a of the drive-side mounting parts 92 of the pole pieces 90, and an upper cylinder part 94 having a cylindrical recess 94a into which the drive device 24G is housed on the side reverse to the lower cylinder part 93. The wound covering sheet 30G and the hard cover 83 are integrated in an airtight manner. The hard cover 83 houses the magnetic coupling surfaces 92a of the drive-side mounting parts 92 of the pole pieces 90 in the cylindrical recess 93a, and the wound covering sheet 30 is attached to the patient's body surface 102 so as to cover the pump body 22G and the part of the inflow/outflow cannula 21 exposed on the body surface together. Then, the drive device 24G is inserted into the cylindrical recess 94a of the hard cover 83. As a result, the driven magnet 56 of the impeller 14G in the pump body 22G and the drive magnet 78 in the drive device 24G are magnetically coupled via the pole pieces 90.

That is, when the drive magnet 78 rotates by the rotation of the motor mechanism 62 in the drive device 24G, two magnetic poles of the drive magnet 78 alternately face the magnetic coupling surfaces 92a of the pair of pole pieces 90. Therefore, the polarities of the pair of pole pieces 90 are alternately switched, and the resultant magnetic force is transmitted to the driven magnet 56. As a result, the driven magnet 56 rotates and the fin 58 of the impeller 14G also rotates, so that blood flowing in through the blood inlet port 38 is sent to the blood outlet port 40. A clearance between the outer periphery of the impeller 14G and the inner periphery of the large part 41 of the housing 32 is formed as a dynamic pressure bearing clearance 86a, and the impeller 14G is supported so as to float and rotate in the radial direction by the dynamic pressure. The impeller 14G is held at a substantially fixed position in the direction of the rotation axis by a passive magnetic bearing constituted by the driven magnet 56 and the pole pieces 90.

In this modification, the portion constituting the large part 41 of the housing 32 of the pump body 22G may be formed from a thermal conductive member 87 (thermal conductive member 87a) having high thermal conductivity such as metal, ceramic, or carbon fiber reinforced resin. In addition, in the casing 60 of the drive device 24G, the wall part that is to face the pump body 22G may also be formed from a thermal conductive member 87b. Further, the portion of the hard cover 83 between the pump body 22G and the drive device 24G may also be formed from a thermal conductive member 87c (particularly a material that prevents generation of eddy current). When the drive device 24G is inserted into the cylindrical recess 94a of the hard cover 83, at least a part of the drive device 24G comes into contact with the hard cover 83 formed from a material having high thermal conductivity.

With the configuration described above, in the extracorporeal-circulation blood pump device 10G, the pump body 22G in which the impeller 14G having a small diameter and long in the direction of the rotation axis rotates at a higher speed can be placed on the body surface 102 without increasing the height of the pump body 22G protruding from the body surface 102. In addition, the drive device 24G can be mounted and removed in the direction perpendicular to the body surface 102, and the pump body 22G can be more easily and reliably covered with the wound covering sheet 30G. Heat generated by the drive device 24G is guided to the pump body 22G from a part of the casing 60 of the drive device 24G formed from a member having high thermal conductivity via the hard cover 83 and the pole pieces 90. The guided heat is further radiated to blood flowing through the pump body 22G via the large part 41. Thus, the drive device 24G is cooled.

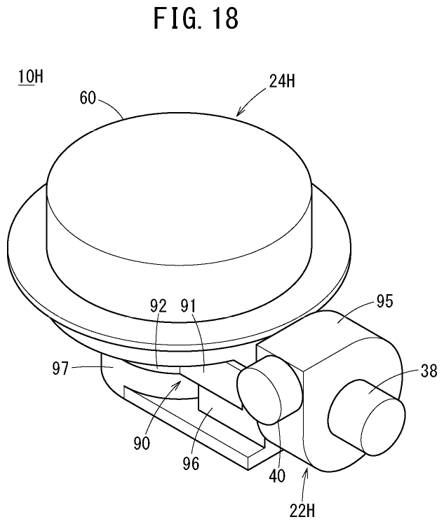

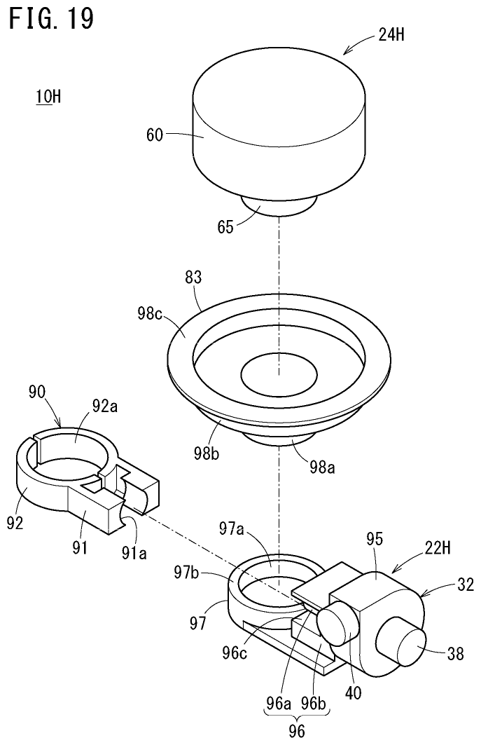

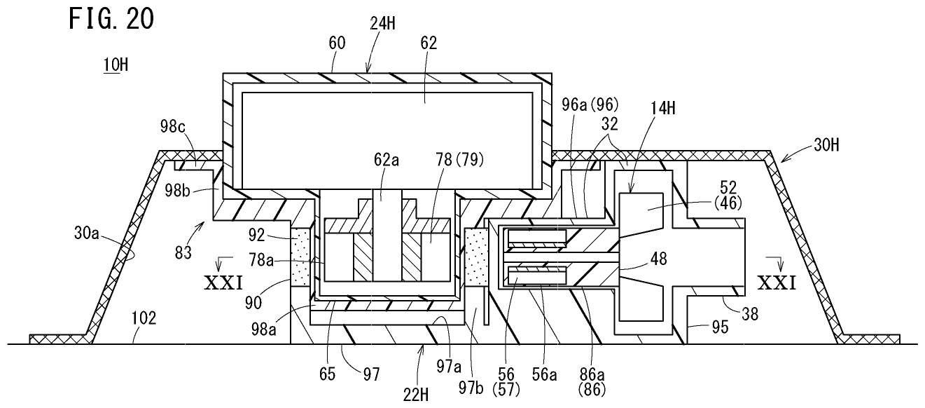

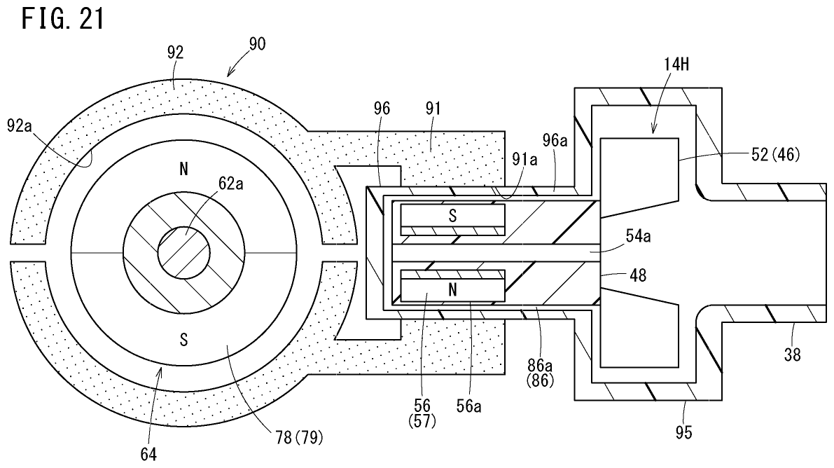

An extracorporeal-circulation blood pump device 10H according to the eighth modification shown in FIGS. 18 to 21 is different from the extracorporeal-circulation blood pump devices 10 and 10A to 10G in that a pump body 22H is of a centrifugal type, and the rotation axis of an impeller 14H is perpendicular to the rotation axis of a motor mechanism 62 of a drive device 24H. The pump body 22H is disposed so that the rotation axis of the impeller 14H is parallel to the body surface 102, as in the extracorporeal-circulation blood pump device 10G according to the seventh modification.

The motor mechanism 62 of the drive device 24H has a shaft 62a of the rotation axis perpendicular to the rotation axis of the impeller 14H, and a drive magnet 78 is fixed to the shaft 62a. The drive magnet 78 is magnetically coupled to a driven magnet 56 (magnetic body) in the impeller 14H via a pair of pole pieces 90 made of a soft magnetic material.

The pump body 22H has a first portion 95 configured in the same manner as the first portion 88 of the pump body 22F according to the sixth modification, and a second portion 96 continuous with the side of the first portion 95, and further has a third portion 97 continuous with the side of the second portion 96. The first portion 95 includes a blood inlet port 38 and a blood outlet port 40 connected to an inflow/outflow cannula 21 inserted into a blood vessel in the body. The first portion 95 houses a fin 52 of the impeller 14H.

The housing 32 constituting the second portion 96 includes a cylindrical part 96a that houses a driven rotating structure section 48 of the impeller 14H in a rotatable manner, and guide bodies 96b provided on upper and lower parts of the cylindrical part 96a and guiding insertion of pole pieces 90. The driven rotating structure section 48 holds a driven magnet 56 that is magnetized with two magnetic poles and has a magnetic coupling surface 56a on the outer periphery. The outer periphery of the cylindrical part 96a is sandwiched between the upper and lower guide bodies 96b, whereby a groove 96c that can be engaged with the pair of pole pieces 90 is formed.

On the other hand, the third portion 97 constitutes a mounted part on which the hard cover 83 of the wound covering sheet 30H and the drive device 24H is mounted in a detachable manner, while supporting the pole pieces 90. The third portion 97 has a cylindrical wall 97b that defines a mounting recess 97a on the inside, and the cylindrical wall 97b is formed to have a height capable of supporting the lower side of the pole pieces 90 inserted into the cylindrical part 96a of the second portion 96.

The pair of pole pieces 90 respectively includes drive-side mounting parts 92 formed in a semi-annular shape, and body-side mounting parts 91 that protrude from the drive-side mounting parts 92 in a predetermined direction. The body-side mounting parts 91 protrude in a direction close to each other at the protruding ends, and have magnetic coupling surfaces 91a that are magnetically coupled to the driven magnet 56 in the impeller 14H on the surfaces facing the cylindrical part 96a of the housing 32. The magnetic coupling surfaces 91a are formed in an arc shape having a central axis coaxial with the rotation axis of the impeller 14H. The drive-side mounting parts 92 have magnetic coupling surfaces 92a perpendicular to the central axis of the magnetic coupling surfaces 91a. That is, the magnetic coupling surfaces 92a of the drive-side mounting parts 92 are also perpendicular to the rotation axis of the impeller 14H, and are magnetically coupled with the drive magnet 78 that is magnetized with two magnetic poles in the drive device 24H and that has a magnetic coupling surface 78a on the outer periphery thereof. In the state where the magnetic coupling surfaces 91a of the body-side mounting parts 91 of the pole pieces 90 are inserted and fitted into the groove 96c formed in the housing 32, the drive-side mounting parts 92 are supported by the cylindrical wall 97b (pole piece base) of the third portion 97.

On the other hand, the hard cover 83 of the wound covering sheet 30H includes a first projecting part 98a which is cylindrical and on which the magnetic coupling surfaces 92a of the drive-side mounting parts 92 of the pole pieces 90 can be mounted on the outer peripheral surface thereof, and a second projecting part 98b which is cylindrical and capable of housing inside the cylindrical projecting part 65 of the drive device 24H. The wound covering sheet 30H is airtightly integrated with a flange 98c extending radially outward from the second projecting part 98b of the hard cover 83.

Inside the wound covering sheet 30H in the covering state, the magnetic coupling surfaces 92a of the drive-side mounting parts 92 of the pole pieces 90 are positioned to face the first projecting part 98a of the hard cover 83. Further, the wound covering sheet 30H is attached to the body surface 102 so as to cover the pump body 22H and a part of the inflow/outflow cannula 21 exposed on the body surface together. Then, the cylindrical projecting part 65 of the drive device 24H is inserted into the first projecting part 98a of the hard cover 83 integrated with the wound covering sheet 30H.

As a result, the driven magnet 56 in the impeller 14H in the pump body 22H and the drive magnet 78 in the drive device 24H are magnetically coupled via the pole pieces 90. Due to the rotation of the motor mechanism 62 in the drive device 24H, the drive magnet 78 rotates, and the driven magnet 56 in the impeller 14H rotates with the rotation of the drive magnet 78 via the pole pieces 90. Thus, the fin 52 rotates, whereby blood flowing in through the blood inlet port 38 is sent through the blood outlet port 40. A clearance between the outer periphery of the driven rotating structure section 48 and the cylindrical part 96a of the housing 32 is formed as a dynamic pressure bearing clearance 86a, and the impeller 14H is supported so as to float and rotate in the radial direction by the dynamic pressure. The impeller 14H is held at a substantially fixed position in the direction of the rotation axis by a passive magnetic bearing constituted by the driven magnet 56 in the driven rotating structure section 48 and the pole pieces 90.

In the extracorporeal-circulation blood pump device 10H configured as described above, the impeller 14H which is long in the direction of the rotation axis and has a small diameter rotates at a higher speed. Further, the drive device 24H can be positioned as close to the body surface 102 as possible, and can be mounted and removed in the direction perpendicular to the body surface 102. Thus, the pump body 22H can be more easily and reliably covered with the wound covering sheet 30H. In the eighth modification, heat generated by the drive device 24H can also be radiated to blood by using a material having high thermal conductivity for the portion of the drive device 24H in contact with the pole pieces 90, the portion of the housing 32 of the pump body 22H in contact with the pole pieces 90, and at least a portion of the hard cover 83.

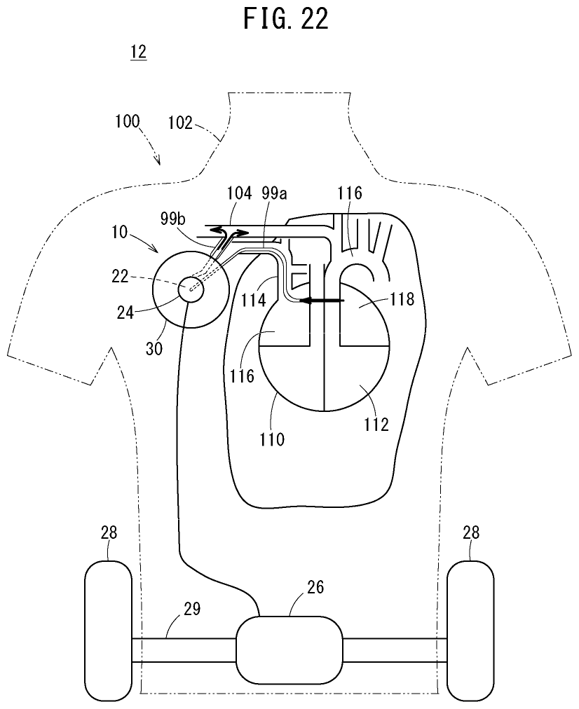

The external ventricular assist device 12 is not limited to have the configuration in which the catheter 16 is inserted and placed in the subclavian artery 104 of the patient 100 as in the above embodiment, and the circulation circuit can be formed in various ways. For example, as in a first usage example shown in FIG. 22, the external ventricular assist device 12 can be configured such that, in place of the catheter 16, a blood removal tube 99a and a blood supply tube 99b are independently connected to any one of the extracorporeal-circulation blood pump devices 10 and 10A to 10H. Hereinafter, an example in which the extracorporeal-circulation blood pump device 10 is used will be described as a typical example.

The blood removal tube 99a is inserted into the subclavian vein 114 from the body surface 102, and further inserted into the left atrium 118 via the right atrium 116 and placed therein. On the other hand, the blood supply tube 99b is inserted into the subclavian artery 104 from the body surface 102 and placed therein.

The wound covering sheet 30 of the extracorporeal-circulation blood pump device 10 covers the locations where the blood removal tube 99a and the blood supply tube 99b are inserted and the pump body 22 together. Thus, the wound covering sheet 30 can firmly fix the extracorporeal-circulation blood pump device 10 and the two tubes 99a and 99b in the same manner as the case where the catheter 16 is placed. Further, when the blood removal tube 99a and the blood supply tube 99b are placed in this way, the extracorporeal-circulation blood pump device 10 can also provide satisfactory flow of blood toward the subclavian artery 104 from the left atrium 118.

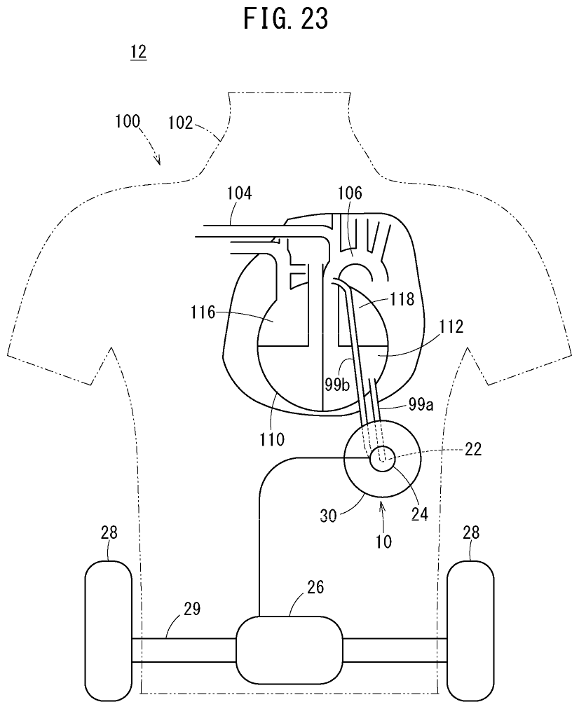

Further, the external ventricular assist device 12 may be configured such that any of the extracorporeal-circulation blood pump devices 10 and 10A to 10H is placed on the body surface 102 near the area below the heart 110, as in a second usage example shown in FIG. 23. In this case, the external ventricular assist device 12 can be configured such that, from the body surface 102 near the location where the external ventricular assist device 12 is placed, the blood removal tube 99a is inserted into the left ventricle 112 and the blood supply tube 99b is inserted into the aortic arch 106.

It is to be noted that the present invention is not limited to the abovementioned embodiment, and various modifications are possible without departing from the spirit of the invention. For example, the extracorporeal-circulation blood pump devices 10 and 10A to 10H are not limited to a centrifugal type or an axial flow type, and may be configured as a mixed flow pump. Alternatively, a pump that circulates liquid by rotating an impeller of another type, such as a spiral flow pump (see PCT publication W02008/053818A1), may also be used. In addition, the extracorporeal-circulation blood pump devices 10 and 10A to 10H are used not only as the external ventricular assist device 12 for assisting the blood circulation of a patient with reduced cardiac function outside the body, but also for increasing the vascular diameter by being connected to peripheral blood vessels, as disclosed in U.S. Pat. No. 9,539,380.

What is claimed is:

1. An extracorporeal-circulation blood pumping apparatus comprising:
a pump body to which a blood removal tube for removing blood from a patient and a blood supply tube for supplying the blood to the patient configured to be connected, and which houses an impeller having inside a body-side magnetic coupling section in a rotatable manner;
a drive device detachably mounted to the pump body and having inside a drive-side magnetic coupling section which includes a rotatable permanent magnet or a stator coil generating a rotating magnetic field, wherein the impeller is rotatable by magnetic coupling between the body-side magnetic coupling section and the drive-side magnetic coupling section; and
a covering sheet configured to affix the pump body to a body surface of the patient, wherein the covering sheet extends radially outward with respect to the pump body and is capable of being affixed to both the pump body and at least a location where the blood removal tube and the blood supply tube are inserted into the body surface, when being attached.

2. The extracorporeal-circulation blood pumping apparatus according to claim 1, wherein the covering sheet is disposed between the pump body and the drive device when the drive device and the pump body are assembled together.

3. The extracorporeal-circulation blood pumping apparatus according to claim 2, wherein the covering sheet has a central opening in which one of the pump body or the drive device is disposed to pass therethrough, and the one of the pump body and the drive device disposed in the opening is mounted to the other in a detachable manner.

4. The extracorporeal-circulation blood pumping apparatus according to claim 3, further comprising:
a hard cover made of a hard material configured to pass through the opening;
wherein the hard cover has a shape configured to conform to the pump body and the drive device; and
wherein the covering sheet is airtightly integrated with the hard cover.

5. The extracorporeal-circulation blood pumping apparatus according to claim 1, wherein the covering sheet is fixed to the pump body.

6. The extracorporeal-circulation blood pumping apparatus according to claim 1, wherein the impeller is provided with a driven rotating structure section including the body-side magnetic coupling section, the impeller being supported so as to float and rotate in a radial direction by a dynamic pressure bearing formed between an outer periphery of the driven rotating structure section and an inner periphery of a housing that houses the driven rotating structure section; and
wherein the driven rotating structure section is held at a substantially fixed position in a direction of a rotation axis by a magnetic bearing formed by the body-side magnetic coupling section and the drive-side magnetic coupling section.

7. The extracorporeal-circulation blood pumping apparatus according to claim 6:
wherein the pump body is placed in such a way that a rotation axis of the driven rotating structure section is parallel to the body surface; and
wherein the drive-side magnetic coupling section of the drive device is a permanent magnet that rotates around a rotation axis disposed coaxial with the rotation axis of the driven rotating structure section.

8. The extracorporeal-circulation blood pumping apparatus according to claim 6:
wherein the pump body is placed in such a way that a rotation axis of the driven rotating structure section is parallel to the body surface, and
wherein the drive-side magnetic coupling section of the drive device is a permanent magnet that rotates around a rotation axis perpendicular to the rotation axis of the driven rotating structure section.

9. The extracorporeal-circulation blood pumping apparatus according to claim 1, further comprising:
a control unit that is electrically connected to the drive device and controls driving of the drive device;
a battery capable of supplying electric power to the drive device and the control unit; and
a storage bag or band capable of holding the control unit and the battery together in a portable manner.

10. An extracorporeal-circulation blood pumping apparatus comprising:
a pump body to which a blood removal tube for removing blood from a patient and a blood supply tube for supplying the blood to the patient configured to be connected, and which houses an impeller having inside a body-side magnetic coupling section in a rotatable manner;
a drive device detachably mounted to the pump body and having inside a drive-side magnetic coupling section which includes a rotatable permanent magnet or a stator coil generating a rotating magnetic field; and
a covering sheet configured to affix the pump body to a body surface of the patient, wherein the covering sheet has an opening in which the pump body or the drive device is disposed to pass therethrough, and one of the pump body and the drive device disposed in the opening so as to pass through the opening is mounted to the other in a detachable manner, and wherein the covering sheet includes an adhesive layer on a side of the covering sheet to adhere to the pump body and to the body surface of the patient.

11. The extracorporeal-circulation blood pumping apparatus according to claim 10, wherein the adhesive layer is comprised of a gel adhesive, a hydrocolloid, a silicone adhesive, or an acrylic adhesive.

12. The extracorporeal-circulation blood pumping apparatus according to claim 10, wherein the adhesive layer is comprised of a disinfectant solution.

13. A method of mounting an extracorporeal-circulation blood pumping apparatus onto a skin surface of a patient, comprising the steps of:
attaching a blood removal tube to a pump body, wherein the blood removal tube receives blood from the patient via a respective insertion location on the skin surface, wherein the pump body houses an impeller having inside a body-side magnetic coupling section in a rotatable manner;
attaching a blood supply tube to the pump body, wherein the blood supply tube returns blood to the patient via a respective insertion location on the skin surface;
disposing the pump body on the skin surface;
adhesively affixing a covering sheet to the pump body and to the skin surface to cover the respective insertion locations, wherein the covering sheet extends radially outward with respect to the pump body; and
detachably mounting a drive device to the pump body, wherein the drive device contains a drive-side magnetic coupling section which includes a rotatable permanent magnet or a stator coil generating a rotating magnetic field, wherein the impeller is rotatable by magnetic coupling between the body-side magnetic coupling section and the drive-side magnetic coupling section.

14. The method according to claim 13, wherein the covering sheet carries an adhesive layer which is comprised of a gel adhesive, a hydrocolloid, a silicone adhesive, or an acrylic adhesive.

15. The method according to claim 14, wherein the adhesive layer is comprised of a disinfectant solution.
